(19) United States
(12) Patent Application Publication
(10) Pub. No.: US 2010/0268051 A1
(43) Pub. Date: Oct. 21, 2010

(54) SYSTEM AND METHOD FOR WELLNESS MONITORING IN A VEHICLE

(75) Inventors: Krishnaswamy Venkatesh Prasad, Ann Arbor, MI (US); Mark Schunder, Dearborn, MI (US); Thomas J. Giuli, Ann Arbor, MI (US)

Correspondence Address: BROOKS KUSHMAN P.C./FGTL, 1000 TOWN CENTER, 22ND FLOOR, SOUTHFIELD, MI, 48075-1238, US

(73) Assignee: FORD GLOBAL TECHNOLOGIES, LLC, Dearborn, MI (US)

(21) Appl. No.: 12/424,826

(22) Filed: Apr. 16, 2009

(51) Int. Cl. A61B 5/145; A61B 5/00; G06F 19/00

(52) U.S. Cl. 600/365; 600/300; 701/36

(57) ABSTRACT

A vehicle-based system interfaces with at least one wellness device to track and monitor various aspects of user wellness. The aspects include, but are not limited to, heart rate, glucose levels, distance traveled, pace, body temperature, etc. The vehicle-based system may be capable of reporting results to a remote server and to the user directly. The vehicle-based system may further be capable of recommending courses of action based on analysis of the results.

### TECHNICAL FIELD

The illustrative embodiments relate generally to human wellness monitoring in conjunction with a vehicle-based computing system.

### BACKGROUND

Home wellness monitoring is a booming business. Existing wellness systems include a variety of wireless devices that can measure different user data. The measurements can vary, and include, but are not limited to, blood pressure, pulse, weight, blood sugar, etc.

In present wellness solutions, the wireless devices can broadcast signals to various hubs, where the signals are then sent from a PC or over a telephone to a caregiver. The signals can also be stored on a PC hard disk drive for patient review.

Wired solutions also exist, again transferring data over a phone or other internet connection to a remote monitoring location, where the data can be reviewed by a care provider.

In addition to tracking critical health metrics and taking preventative measures to stave off major catastrophes, wellness also includes the measurement and tracking of general health related metrics. For example, Nike and Apple have combined to produce a sports kit that can track the duration of a workout, the distance traveled, the pace, the calories burned, and various other metrics. This information can then be analyzed by a user to track workout statistics.

### SUMMARY OF ILLUSTRATIVE EMBODIMENTS

Illustrative embodiments include a wellness monitoring system comprising for use in a vehicle. The system includes, among other things, a vehicle-based processor, to control the monitoring system. It may also include one or more storage locations to store data for use by the processor, and a transceiver, in communication with the processor, to receive and send data to and from the processor. An output, either audio (such as speakers), visual (such as an LCD display), or both, may also be included.

In at least one illustrative embodiment, the transceiver, upon receiving an alert signal from a wellness device, transmits the signal to the processor. The processor then may instruct the system to automatically locate at least one point of interest relevant to the received signal. The system may also be instructed to output directions to the point of interest to which a vehicle driver may wish to travel after a condition causing the signal has occurred.

For example, the signal could be a low blood-sugar signal, and the system could automatically locate a convenience store, such as a 7-11 and/or a hospital. The user could be provided with directions to both, or at least an option to choose which directions were desired.

In at least another illustrative embodiment, the wellness device continually transmits a signal to the vehicle, and the vehicle-based processor analyzes the signal.

For example, the processor could compare the signal to a predetermined or user-entered threshold, to see if an emergency condition is present.

Or, the wellness device could be a fitness device, transmitting continually, periodically, or at one or a few times. In the case where the wellness device is a fitness device, the processor may be in communication with the fitness device through the transceiver, and may recommend a route for, say, an exercise routine.

The processor may also know when the route should be finished, and analyze conditions, such as temperature, in the car before the user is due to complete the routine. If the conditions in the car are outside a desired range, the processor may, for example, instruct the running of heating or air conditioning in order to make the environment in the car suitable for a post-workout user.

### BRIEF DESCRIPTION OF THE DRAWINGS

Other objects, aspects and characteristics of the illustrative embodiments will become apparent from the following detailed description of exemplary embodiments, when read in view of the accompanying drawings, in which:

FIG. 1 shows an exemplary illustrative system capable of communication with a wellness device;

FIG. 2 depicts a flow diagram that illustrates an exemplary illustrative method for executing wellness-related functions via the vehicle-based system;

FIGS. 3A and 3B show illustrative examples of simultaneous connection between a vehicle-based system and multiple wellness devices; and

FIG. 4 shows an illustrative example in which the vehicle-based system can continuously monitor one or more wellness parameters of each respective wellness device and present select parameters to a user via the user interface.

### DETAILED DESCRIPTION OF THE EMBODIMENT

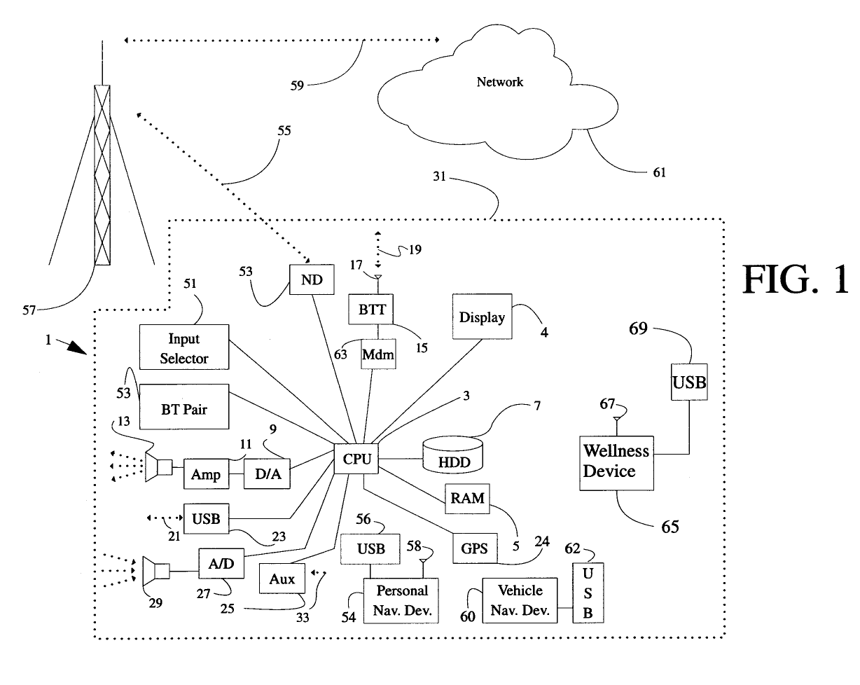

FIG. 1 illustrates system architecture of an illustrative onboard computing and communication system usable for delivery of directions to an automobile. A vehicle enabled with a communication and entertainment system (VCES) may contain a visual front end interface 4 located in the vehicle. The user may also be able to interact with the interface if it is provided, for example, with a touch sensitive screen. In another illustrative embodiment, the interaction occurs through audible speech and speech synthesis.

In the illustrative embodiment 1 shown in FIG. 1 a processor 3 controls the operation of the system. Provided within the vehicle itself, the processor allows onboard processing of commands and routines. Further, the processor is connected to both temporary 5 and permanent storage 7. In this illustrative embodiment, the temporary storage is random access memory (RAM) and the permanent storage is a hard disk drive (HDD) or flash memory.

The processor is also provided with a number of different inputs for the user to interface with the processor. In this illustrative embodiment, a microphone 29, an auxiliary input 25 (for input 33), a USB input 23, a GPS input 24 and a BLUETOOTH input 15 are all provided. An input selector 51 is also provided, to allow a user to swap between various inputs. Input to both the microphone and the auxiliary connector is converted from analog to digital by a converter 27 before being passed to the processor.

Outputs to the system can include, but are not limited to, a visual display 4 and a speaker 13 or stereo system output. The speaker is connected to an amplifier 11 and receives its signal from the processor 3 through a digital-to-analog converter 9. Output can also be made to a remote BLUETOOTH device (not shown) or a USB device (not shown) along the bi-directional data streams shown at 19 and 21 respectively.

In one illustrative embodiment, the system 1, uses the BLUETOOTH transceiver 15 to communicate 17 with a user's nomadic device 53 (e.g., cell phone, smart phone, PDA, etc.). The nomadic device can then be used to communicate 59 with a network 61 outside the vehicle 31 through, for example, communication 55 with a cellular tower 57.

Pairing a nomadic device 53 and the BLUETOOTH transceiver 15 can be instructed through a button 53 or similar input, telling the CPU that the onboard BLUETOOTH transceiver will be paired with a BLUETOOTH transceiver in a nomadic device.

Data may be communicated between CPU 3 and network 61 utilizing a data-plan associated with nomadic device 53. Alternatively, it may be desirable to include an onboard modem 63 in order to transfer data between CPU 3 and network 61 over the voice band. In one illustrative embodiment, the processor is provided with an operating system including an API to communicate with modem application software. The modem application software may access an embedded module or firmware on the BLUETOOTH transceiver to complete wireless communication with a remote BLUETOOTH transceiver (such as that found in a nomadic device). Other wireless technology such as, but not limited to, WiFi, could also be used. In another embodiment, nomadic device 53 includes a modem for voice band or broadband data communication. In the data-over-voice embodiment, a technique known as frequency division multiplexing may be implemented when the owner of the nomadic device can talk over the device while data is being transferred. At other times, when the owner is not using the device, the data transfer can use the whole bandwidth (300 Hz to 3.4 kHz in one example). If the user has a data-plan associated with the nomadic device, it is possible that the data-plan allows for broad-band transmission and the system could use a much wider bandwidth (speeding up data transfer). In still another embodiment, nomadic device 53 is replaced with a cellular communication device (not shown) that is affixed to vehicle 31.

In one embodiment, incoming data can be passed through the nomadic device via a data-over-voice or data-plan, through the onboard BLUETOOTH transceiver and into the vehicle's internal processor 3.

The vehicle-based system can communicate with a personal navigation device 60, having a USB 62 or other connection, an onboard GPS device 24, or remote navigation system (not shown) having connectivity to network 61. Services that can be accessed through the network include, but are not limited to, private communications, emergency services, health and wellness sites, databases, etc.

The vehicle-based computing system may be configured to communicate with one or more wellness devices (generally represented by 65).

A local communication interface of the vehicle-based computing system may communicate with each wellness device's local communication interface 67, 69. As described, such local communication can be achieved wirelessly (e.g., Bluetooth) and/or via cable(s) (e.g., USB).

Wellness device 65 may be a health monitoring device, a fitness monitoring device, or another type of wellness device. A health monitoring device, as used in this document, generally refers to a wellness device that can monitor one or more health parameters (e.g., glucose level) associated with the user's health. Examples of health monitoring devices include, but are not limited to, heart monitors and glucose monitors. An example of a commercially available health monitoring device is the GUARDIAN RT GLUCOSE MONITORING SYSTEM available from Medtronic, Inc. (www.medtronic.com). A wellness device may also be a fitness monitoring device, as used in this document, and generally refers to a wellness device that is configured to monitor one or more fitness parameters (e.g., running speed) associated with the user while he or she is engaged in a fitness activity, such as walking, running, swimming, or the like. An example of a commercially available fitness device is Nike PLUS®. Other types of wellness devices include, non-exhaustively, pedometers, bicycle computers, and the like.

Each wellness device 65 may take a number of forms. In one or more illustrative embodiments, wellness device 65 is a portable device that can be worn by, coupled to, or proximate to a user. Further, according to one or more illustrative embodiments, a wellness device 65 is a fixed device that can be mounted in the motor vehicle 31, for example, coupled to a vehicle seat. Alternatively, a wellness device 65 can be a combination of portable and fixed devices.

As described in greater detail below, health and wellness devices may include (but are not limited to) devices such as blood glucose monitors, running monitors, pedometers, bicycle monitors, heart rate monitors and the like.

Wellness devices may be equipped with, or be equipped to interface with, a communications interface to communicate information to/from processor 67, 69, or data storage associated therewith. The communications interface may include a BLUETOOTH transceiver or a USB interface for wireless or wired communication with processor 3.

In the case of an exemplary BLUETOOTH-enabled wellness device, a pairing mode may be entered into at the vehicle-based system and the wellness device 65. Upon pairing, vehicle-based system, and more particularly processor 3, may be programmed and configured to automatically detect the presence of, and establish communication with, a wellness device within the vicinity or passenger compartment of vehicle 31.

For example, when a runner using a NIKE PLUS device configured for communication with a vehicle-based system returns to the vehicle 31 following a run, the vehicle-based system may automatically detect the presence of the NIKE PLUS device. The vehicle-based system may initiate communication with the device to download to memory 107 or storage 7 statistics collected or calculated by the NIKE PLUS device during the run. As described in greater detail below, portions of the downloaded information, or analysis of the downloaded information, among other things, may be reported to the vehicle occupant(s) via a user interface.

The wellness device(s) 65 can have a unique identifier, such as a hardware identifier, a firmware identifier, a MAC address or the like stored to a computer readable medium (not shown). For example, one of the wellness devices 65 may have a serial number unique serial number “A28B392O.” This identifier may be communicated to vehicle-based system and processor 3 to identify and correlate information such as a registered owner or user of the device and predefined owner/user preference settings. Owner/user names, preferences, etc. may be stored locally in persistent memory 7, or remotely at the server and accessible to vehicle-based system via the wide area network.

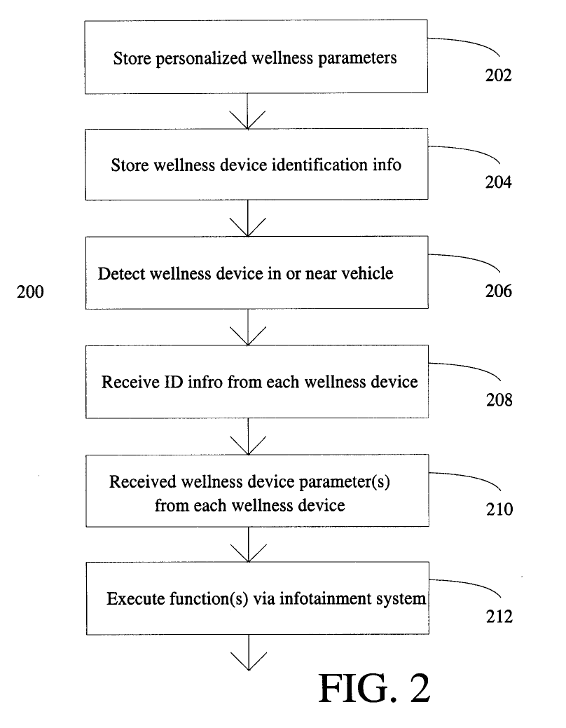

FIG. 2 depicts a flow diagram 200 that generally illustrates an exemplary illustrative method for executing wellness-related functions via the vehicle-based system in response to detecting one or more wellness devices 65 and/or receiving data from the wellness device(s) 65. Each step in the method is represented by a respective block 202-212. Steps may be added to, removed from, and/or reordered in the method.

Block 202 depicts a step in which wellness information relating to one or more users is stored, for example, to the server(s) at a remote site. For example, the server can host a website (e.g., http://www.syncmyride.com), and a user can access the website via the Internet to store the wellness information, predefined preferences, settings, etc. to the server. The wellness information can include any information relating to a user's health and/or wellness. For example, the information may include: user's name or photographic image, a desired range of glucose levels or glucose alarm levels, an optimal heart rate or heart rate alarm levels, display or playback preferences, etc.

Block 204 depicts a step in which identification information relating to each wellness device 65 is stored, for example, to a server, a computer readable medium (not shown) in the vehicle-based system, or a combination thereof. For example, the vehicle-based system can receive or retrieve a device identifier, user name and preference settings for a plurality of different wellness devices 65. Additional information may also be stored at this step, such as a preferred doctor and/or hospital, known medical conditions, an urgent care provider, and any other suitable information.

Block 206 depicts a step in which one or more wellness devices 65 are detected in or near the motor vehicle 31. For example, if the vehicle-based system and the wellness device(s) 65 have Bluetooth compatibility, the vehicle-based system can detect a given wellness device 65 once it enters a given spatial distance from the vehicle-based system. As another example, if the vehicle-based system and the wellness device 65 are configured to connect to one another via a transmission line (e.g., a USB cable), the vehicle-based system can detect a given wellness device 65 once it is plugged in to the vehicle-based system. Block 208 depicts a step in which the vehicle-based system receives and stores identifiers from each wellness device 65. For example, each identifier can be a unique serial number. In one or more illustrative embodiments, user identification and preference information for the identified device are retrieved locally from persistent memory 7, or remotely from the web server to govern or enhance the display or playback of information to the vehicle occupant(s). User identification and preference information may have been input locally at vehicle-based system, over the Internet to the web server at a web site, such as www.syncmyride.com, or in another suitable fashion.

Block 210 depicts a step in which the vehicle-based system receives and stores one or more wellness parameters from each wellness device 65. For example, the wellness device 65 may be a glucose monitor configured to communicate a user's glucose level via its local communication interface, either at spaced time intervals or upon an event (e.g., an exceedingly low glucose level). Here, the vehicle-based system can receive data defining the user's glucose level via the local communication interface. Additionally, the system could receive personal information stored, for example, at step 204.

Block 212 depicts a step in which the vehicle-based system executes one or more functions based, at least in part, on the stored data relating to the wellness device(s) 65, user preferences, and/or one or more wellness parameters from the wellness device(s) 65. As an example, assume that one of the wellness device(s) 65 is a glucose monitor, that user wellness information, including a user's acceptable glucose range, has been stored to a vehicle database or to a server at a remote site (e.g., a website), and that the glucose monitor has been detected by the vehicle-based system (e.g., via a BLUETOOTH connection). In this exemplary scenario, the vehicle-based system can receive a glucose level from the glucose monitor. The signal may come, for example, once (e.g., upon detection of the glucose monitor), at spaced time intervals, at other user-defined times, etc. The vehicle-based system can then compare the received glucose level to the predefined glucose range for the identified device, and if the glucose level is outside of the glucose range, notify the vehicle occupant(s). The notification may visually or audible identifies a person's name associated with the particular glucose monitoring device using a predefined name attribute associated with the device's unique identification number. As explained above, this association, and others, may be done locally at the vehicle-based system, or remotely over the Internet via a web server.

In one or more illustrative embodiments, processor 3 may be configured to automatically generate a set of emergency points of interest located in the vicinity of automobile 31 in the event a glucose level of a vehicle occupant exceeds a permissible range. The emergency points of interest may be identified based on a current location obtained from a global positioning or navigation system (not shown) within the vehicle. Alternatively, vehicle-based system may be configured to query the server or a local a GPS or navigation-enabled cellular telephone for location and/or emergency points of interest. Emergency points of interest may include, but are not limited to, hospitals, urgent care centers, fire houses, restaurants, and convenience stores.

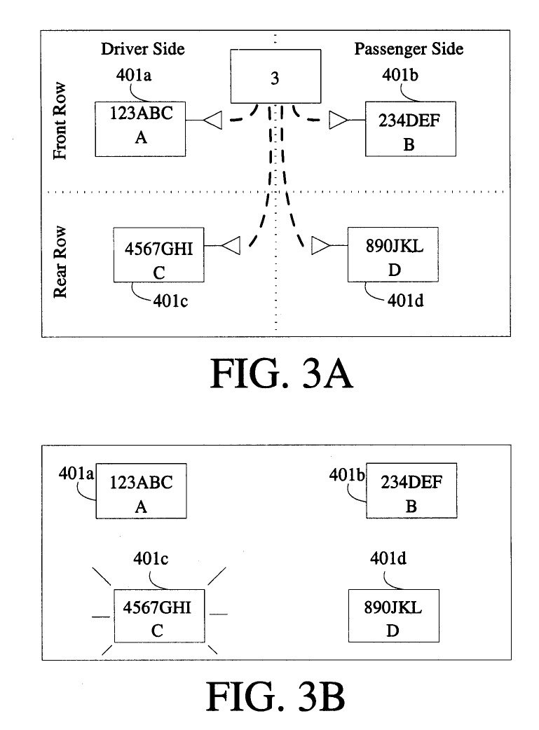

In one or more illustrative embodiments, an exemplary non-limiting illustration of which is shown in FIG. 3A, vehicle-based system may simultaneously communicate, as necessary, with multiple wellness monitoring devices in the vehicle passenger compartment (as illustrated) or in the immediate vicinity of the vehicle. Predefined pairings or correlations between occupant name and vehicle ID (e.g. 123ABC for driver of vehicle 31) stored in a vehicle database or at a web server are one way to enable the vehicle-based system 3 to display or otherwise output to the passenger compartment the specific status or condition of multiple occupants. This output may occur in response to an event as described above, at regular predefined time intervals, upon an occupant's query to the vehicle-based system, or at any other suitable moment.

For example, if vehicle 31 includes father Jim at location “A,” mother Susan at location “B,” son John at location “C,” and daughter Kathy at location “D,” and if each occupant carries with him or her a personal glucose monitoring device 401a-d previously paired with vehicle-based system including user identification information, processor 3 may be programmed and configured to automatically generate the visual display 400 illustrated in FIG. 3B. If a passenger's glucose falls below a predefined level, the icon 402 corresponding to that passenger may change state such as illustrated by icon 402c, or some other indication may be provided. The particular levels at which these changes occur may be predefined, for each occupant, at the vehicle-based system, through the web server (e.g. www.syncmyride.com), or in any other suitable fashion.

Although only two rows are shown in the illustrative embodiment of 3A, it is, of course, possible to have a vehicle with more than two rows, and a suitable configuration and detection could be provided for such a vehicle as well. The embodiments hereof are not intended to be limited to a vehicle having a particular number of rows.

In at least one illustrative embodiment, real-time (or near real-time) health levels may be visually displayed at interface 400 for one or more vehicle occupants, as illustrated in FIG. 3B. Alternatively, an occupant may queue the vehicle-based system, by touch or audible question, to determine a particular occupant's level. For example, mother Susan may audibly ask vehicle-based system what John's glucose level is, and by speech recognition the system may programmatically obtain (either from storage in memory, a database or from the device 402c) the current level. The current level may be reported back visually at interface 400 or audibly, for example, by a text-to-speech module (not shown) in operable communication with processor 3.

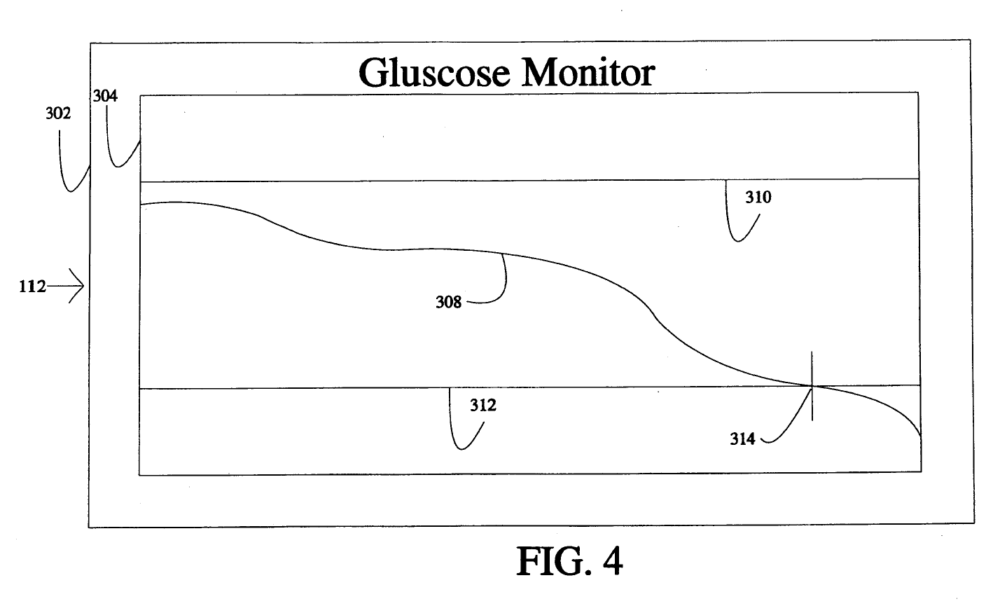

FIG. 4 shows an illustrative example, according to at least one illustrative embodiment, in which the vehicle-based system can continuously monitor one or more wellness parameters of each respective wellness device 65 and present select parameters to a user via the user interface. Here, the user interface includes a display 302 that shows a graph 304 of glucose level along the vertical direction and time along the horizontal direction. The display 302 has a title 306 that associates the graph 302 with a particular user. The information in the title 306 (e.g., Joe Smith) can be stored information that is associated with a wellness device's identifier (e.g., serial number). Referring to the graph 304, a curve 308 represents a user's glucose level over time. Two horizontal lines 310,312 represent upper and lower glucose level limits, respectively. These limits can be representative of data stored, for example, to a server in step 202. In the shown example, when the glucose level decreases below the lower limit 312 at time tₐ 314, the vehicle-based system can suitably alert the user (i.e., via the display, via speakers, etc.) and/or provide a point of interest to the user. In the shown embodiment, the wellness parameter is a glucose level. However, in other illustrative embodiments, any suitable wellness parameter may be monitored and/or presented via the user interface.

Also, according to one or more illustrative embodiments, data collected from a mobile monitoring device during a period of time or during an exercise may be visually and/or audibly presented to a vehicle occupant upon reentry to the vehicle. Processor 3 may be programmed and configured to initiate the playback or a summary of the information automatically upon reentry to the vehicle. Playback/summary may be presented upon occupant request, or based on predefined preference settings made at the vehicle, a web server, through a web site, such as www.syncmyride.com, etc. for the particular mobile device at issue.

For example, upon the return of a runner having a BLUETOOTH-enabled NIKEPLUS device configured for communication with the vehicle-based system, a local BLUETOOTH transceiver in a vehicle-based system may automatically detect the presence of the BLUETOOTH transceiver coupled to or embedded within the NIKEPLUS device. Upon detection and connection, data concerning the current run may be downloaded from the NIKEPLUS device to a memory or storage 7 associated with processor 3. Alternatively, a USB or other wired data connection may be established between the NIKEPLUS device and the vehicle-based system for communicating collected data to the vehicle 31 for storage in memory or a database accessible by processor 3.

Processor 3 may be configured to visually display or playback by speech engine a subset, all or a summary of the information received from the NIKEPLUS device. Users may set preferences at the vehicle or at web server to specify which information to be presented, or the manner in which the data is to be presented. For example, one runner may specify that the runner's starting, maximum and average heart rate be displayed or played back upon reentry to the vehicle. Another runner may only be concerned with her average heart rate, and the amount of time that her heart rate was above a predefined threshold. A third runner may request a graphical display of his heart rate and ambient temperature over the duration of the run. Ambient temperature may be collected by the mobile monitoring device. Alternatively, the ambient temperature may be captured by the vehicle over the duration of the exercise, and coupled with the data received from the mobile device for presentation to the vehicle occupant. Of course, there are a myriad of playback options which depend on the unique preferences of the user and the capabilities of and data collected by the mobile monitoring device.

In another example, the mobile monitoring device is a bicycle computer configured for communication with vehicle-based system. The bicycle computer may include an embedded BLUETOOTH transceiver or an interface for wired data communication with vehicle-based system. Bicycle computers may receive a plurality of inputs during a ride including but not limited to rider heart rate, instantaneous speed, maximum speed and average speed. Average speeds may be calculated over the entire course of a ride, or only during the period of time in which the bicycle is in motion (i.e. not during periods of rest). This data, like data received from the NIKEPLUS device following a run, may be played back to the user or vehicle occupant upon reentry to the vehicle after an exercise. The playback settings may be user-specified and complied with based on the unique identification of the particular mobile device.

It is not uncommon for people to exercise together, each person having his or her own exercise monitoring device. For example, two friends may jog together, each having his or her own NIKEPLUS device. Processor 3 may be programmed and configured to permit the two friends to determine which of their data to display or playback, and in what order. In one or more illustrative embodiments, the display will present icons corresponding to each monitoring device in communicable proximity to vehicle 31. An occupant may physically select one of the icons to initiate display and playback of recorded data from the selected monitoring device. As explained above, the display may include the name of the device's user or owner if that information was input to a vehicle-based system or uploaded to a web server through a website. Alternatively, occupants may present vehicle-based system with voice commands to initiate display and playback of exercise data.

If multiple occupants having exercise monitors engage in a similar exercise, processor 3 may be configured to display received data in a comparative fashion. For example, heart rate over time may be simultaneously plotted on the display for two runners that have run together, showing a comparison of each person's heart rate with respect to his or her running companion. Alternatively, data may be displayed or played back in comparison with, or together with, historical data collected during prior exercises of a similar type. Archival exercise data may be stored in a vehicle database or at a web server for subsequent retrieval. In this illustrative embodiment, a runner's current run statistics may be graphically or audible compared to the runner's statistics from a prior run, or from an average of all or selected prior runs.

Users may specify or otherwise configure options for archival storage of received exercise, health and wellness data at vehicle-based system, or a web server. For example, one user may wish to archive all data for all times for future access, either by vehicle-based system, or over the Internet by web browser for in-home review and analysis. Another user may not want any of his or her exercise, health or wellness data archived anywhere. A third user may only want his exercise data archived at a web server, and not at the vehicle 31. An interface for setting these and other options (such as password access, display parameters, etc.) may be presented to occupants and device owners at the vehicle-based system or on a web page such as www.syncmyride.com at a server accessible through a web browser. Exercisers can even use this data to compete with and compare results to similarly situated other exercisers who also are using the vehicle-based system.

As described above, processor 3 may be in communication with other vehicle systems, typically over a controller area network (CAN) bus or other multiplex vehicle bus. In one or more illustrative embodiments, processor 3 monitors the bus, or otherwise receives a signal, indicating when the vehicle has been placed in any drive gear, such as drive or reverse. Upon receipt of this signal, processor 3 may automatically terminate visual display of exercise, health or wellness data. The display may be reinstated upon placement of the vehicle in park. Alternatively, the displayed information, or a subset thereof, may be played back to vehicle occupant(s) audibly upon placement of the vehicle in a drive gear.

In at least one illustrative embodiment, the vehicle-based system can further suggest to a user one or more items he or she should purchase based, at least in part, on the wellness parameter(s). In the foregoing example, the vehicle-based system can, for example, locate the nearest convenience store, provide directions to the store, and suggest that the person drive to that store and purchase a can of soda if his or her glucose level is sufficiently low.

Additionally, the vehicle-based system can adjust the climate in the vehicle 31 based, at least in part, on information from one or more of the wellness devices 65. As one example, the wellness device 65 is a fitness device, such as the Nike PLUS® and a user of the fitness device returns to the motor vehicle 31 from a fitness activity (e.g., running). Once the vehicle-based system detects the fitness device and receives one or more fitness parameters from it (e.g., total distance ran by the user), the vehicle-based system can adjust the climate in the vehicle-based on information such as, but not necessarily limited to, the fitness parameter(s), the interior climate of the vehicle, and the exterior climate of the vehicle.

Further, the vehicle-based system can delay and/or disable the vehicle's ignition system based, at least in part, on information from one or more of the wellness devices 65. In the foregoing example, if a user's heart rate, for example, exceeds a predetermined threshold (e.g., information stored by the user to the server) the vehicle-based system can disable the ignition system, monitor the user's heart rate, and enable the ignition system when the user's heart rate returns to an acceptable level.

In another illustrative embodiment, a vehicle-based system may recognize that a health or wellness device has entered into communication range, and accordingly communicate with that device. Although devices are typically paired with a vehicle-based system to enable communication, it may be possible to automatically enact a pairing in the case of certain devices. For example, if a passenger wearing a diabetes monitor (blood-sugar) were to enter a vehicle, the vehicle-based system may begin tracking the blood-sugar level of the passenger. Additionally, in the case of, for example, a child, the vehicle-based system may notify the child's parent that the child is in the vehicle, if it is not the “home” vehicle for that child. This can be done through, for example, the network, and allow the parents to track the location and status of the child.

Monitoring of wellness/health devices, and storage of monitoring and other health related information may also be useful in the event of an accident. Passenger medical information can be sent directly to first responders, so they know about allergies, passenger conditions, etc. Real time statistics can also be reported to doctors, hospitals and other medical care providers to monitor passengers.

While certain illustrative embodiments have been illustrated and described, it is not intended that these embodiments illustrate and describe all possible forms. Rather, the words used in this section are words of description rather than limitation, and various changes may be made without departing from the spirit and scope of the invention.

What is claimed:

1. A vehicle-based wellness monitoring system comprising:
a vehicle-based processor, to control the monitoring system;
one or more storage locations to store data for use by the processor;
a transceiver, in communication with the processor, to receive and send data to and from the processor;
an output to provide output information to a user, wherein the transceiver, upon receiving an alert signal from a wellness device, transmits the signal to the processor, and wherein the processor instructs the system to automatically locate at least one point of interest relevant to the received signal and to output directions to the point of interest to which a vehicle driver may wish to travel after a condition causing the signal has occurred.

2. The system of claim 1, wherein the output is an LCD display.

3. The system of claim 1, wherein the output is an audio output.

4. The system of claim 1, wherein the information includes directions to at least a hospital.

5. The system of claim 1, wherein the signal is a low blood sugar signal.

6. The system of claim 5, wherein the information includes directions to at least a convenience store.

7. A vehicle-based wellness monitoring system comprising:
a vehicle-based processor, to control the monitoring system;
one or more storage locations to store data for use by the processor;
a transceiver, in communication with the processor, to receive and send data to and from the processor;
an output to provide output information to a user, wherein the transceiver, upon receiving a signal from a wellness device, transmits the signal to the processor, and wherein the processor determines if an emergency condition is present, wherein if an emergency condition is present, the processor instructs the system to automatically locate at least one point of interest relevant to the received signal and to output directions to the point of interest to which a vehicle driver may wish to travel after a condition causing the signal has occurred.

8. The system of claim 7, wherein the output is an LCD display.

9. The system of claim 7, wherein the output is an audio output.

10. The system of claim 7, wherein the information includes directions to at least a hospital.

11. The system of claim 7, wherein the signal is a blood sugar signal.

12. The system of claim 11, wherein the information includes directions to at least a convenience store.

13. The system of claim 11, wherein the processor compares the signal to a threshold to determine if the emergency condition is present.

14. The system of claim 13, wherein the threshold is user determined.

15. The system of claim 13, wherein the threshold is present.

16. A vehicle-based wellness monitoring system comprising:
a vehicle-based processor, to control the monitoring system;
one or more storage locations to store data for use by the processor;
a transceiver, in communication with the processor, to receive and send data to and from the processor;
wherein the transceiver receives data from and one or more wellness devices and sends the data to the processor to be analyzed.

17. The wellness monitoring system of claim 16, wherein the vehicle-based system is operable to deactivate at least one vehicle component based at least in part on analysis of the information received from the wellness devices.

18. The wellness monitoring system of claim 16, wherein at least one wellness device is a fitness related wellness device, and wherein the vehicle-based system is operable to determine a recommended travel route for an exercise routine.

19. The wellness monitoring system of claim 18, wherein the transceiver transmits information relating to the recommended travel route to the at least one fitness related wellness device.

20. The wellness monitoring system of claim 18, wherein the processor determines approximately when a user should complete the exercise routine, wherein the processor further receives information including the temperature of the vehicle, and at a predetermined time, based at least in part on the determination as to when a user should complete the exercise routine, if the vehicle temperature is outside a recommended range, activate a heating or cooling system to cause the vehicle temperature to adjust to a temperature within the predetermined range.
